(19) United States
(12) Patent Application Publication
(10) Pub. No.: US 2025/0250300 A1
(43) Pub. Date: Aug. 7, 2025

(54) COMPOSITIONS AND METHODS FOR TREATING PSEUDOMYXOMA PERITONEI

(71) Applicant: UNIVERSITY OF SOUTH CAROLINA, COLUMBIA, SC (US)

(72) Inventors: JIE LI, COLUMBIA, SC (US); DAPING FAN, COLUMBIA, SC (US); HEXIN CHEN, IRMO, SC (US); TRACI TESTERMAN, COLUMBIA, SC (US)

(21) Appl. No.: 19/043,664

(22) Filed: Feb. 3, 2025

Related U.S. Application Data
(60) Provisional application No. 63/627,922, filed on Feb. 1, 2024.

(51) Int. Cl. C07K 7/64; A61K 38/00; A61P 35/00; G01N 33/50

(52) U.S. Cl. CPC C07K 7/64; A61P 35/00; G01N 33/5038; G01N 33/5044; A61K 38/00; G01N 2500/10

(57) ABSTRACT

In general, disclosed herein are compositions and methods for treating pseudomyxoma peritonei (PMP). Compounds disclosed herein for treating pseudomyxoma peritonei may include PMP inhibitors of Formula I:

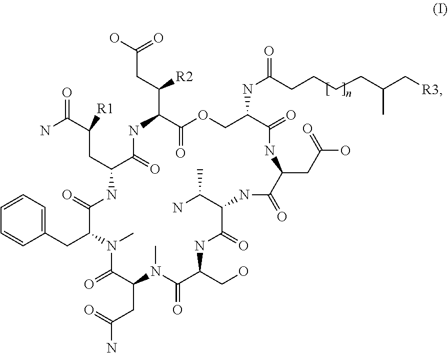

or a pharmaceutically acceptable salt thereof.

### CROSS REFERENCE TO RELATED APPLICATIONS

This application claims the filing benefit of U.S. Provisional Patent Application Ser. No. 63/627,922, filed on Feb. 1, 2024, which is incorporated herein by reference.

### GOVERNMENT SUPPORT CLAUSE

This invention was made with government support under Grant No. R35 GM150565 awarded by National Institutes of Health (NIH). The government has certain rights in the invention.

### BACKGROUND

Natural products play an important role in combating cancer, such as vincristine, paclitaxel, camptothecin, mitomycin C, and actinomycin D for treatment of a variety of cancers in clinic. Despite their success in clinical cancer treatment, natural products are seldom screened against rare cancers, such as pseudomyxoma peritonei (PMP), which originates as an appendiceal neoplasm. Due to its usually poor vascularization and tendency to form floating nodules, PMP spreads easily within the peritoneal cavity, causing mucinous ascites and tumor implants on the surfaces of the peritoneal wall and abdominal organ surfaces. Due to the limited effectiveness of current chemotherapy drugs against PMP, the standard treatment involves a high-risk, intensive surgery spanning more than 10 hours, which poses significant danger to patients in weakened health and leaves them with no viable alternative treatments. PMP not only poses unique diagnostic and therapeutic dilemmas due to its rarity and complex biology, but also lacks a platform for pre-clinical drug testing.

High throughput bioactivity screening has proved effective in discovering bioactive natural products. Although this approach can target desired bioactivities, it lacks the ability to target novel chemistry. This drawback can be complemented by metabolomics, especially when coupled with emerging cheminformatics tools such as Global Natural Product Social Molecular Networking (GNPS), MZmine, and Sum formula Identification by Ranking Isotope patterns Using mass Spectrometry (SIRIUS). When combined with high-throughput screening, these tools can enable precise mining and targeted isolation of bioactive natural products with novel structural features. Another hurdle in natural product-based drug discovery is the unambiguous elucidation of complex chemical structures, particularly when confronted with limited material. This challenge can be addressed by the recent advancement in microcrystal electron diffraction (MicroED), which enables the analysis of microcrystals even in a mixture, offering easier processing when compared to X-ray single crystal diffraction.

As such, a need exists in the art for methods for high-throughput screening and/or identification of compounds that may be useful in treating rare cancers, such as pseudomyxoma peritonei.

### SUMMARY

In general, disclosed herein are pseudomyxoma peritonei (PMP) inhibitors of Formula I:

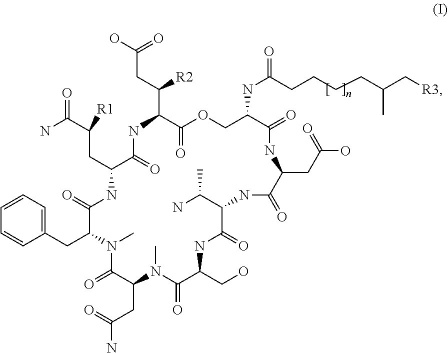

or a pharmaceutically acceptable salt thereof, wherein:

R1 through R3 is independently selected from hydrogen,—OH, or —CH₃; and

n is 6 or 7.

Additionally, disclosed herein are methods for treating pseudomyxoma peritonei. The method may include administering to a subject in need thereof a therapeutically effective amount of a PMP inhibitor or a pharmaceutically acceptable salt thereof.

Also, disclosed herein are methods for screening and/or identifying an inhibitor for treating pseudomyxoma peritonei. The methods may include contacting a stable cell line with an inhibitor; measuring a level of one or more metabolites selected from Table 1; and comparing the measured level of one or more metabolites selected from Table 1 with a level of the one or more metabolites in the cell line before contact with the inhibitor.

Other features and aspects of the present disclosure are discussed in greater detail below.

### BRIEF DESCRIPTION OF THE DRAWINGS

A full and enabling disclosure of the present disclosure is set forth more particularly in the remainder of the specification, including reference to the accompanying figures, in which:

FIG. 1A depicts 16S rDNA-based phylogeny and whole genome-based ANI analysis of PMP498-3.

FIG. 1B depicts Keyence Imaging of PMP501-1 and ABX023-1 cells after co-incubation with the bacteria PMP498-3 and PMP191F. Bacteria inside the PMP cells were stained with carboxyfluorescein diacetate N-succinimidyl ester (CFDA-SE). The actin filaments of PMP cells were stained with phalloidin.

FIG. 1C depicts cell viability assay of PMP498-3 and PMP191F extracts on PMP501-1 and HT-29 cells at the concentration of 10 μg/mL.

FIG. 2A depicts biosynthetic gene cluster of myxomapeptin.

FIG. 2B depicts predicted functions of proteins encoded by genes in the myx cluster.

FIG. 2C depicts chemical structures of myxomapeptins A (1) and B (2).

FIG. 3A depicts crystal violet cell viability assay of myxomapeptin B (2) and 5-fluorouracil (5-FU, positive control) toward PMP, HT-29 and FHC cell lines

FIG. 3B depicts seahorse mitochondrial stress assay of 2 and 5-FU (positive control) toward PMP501-1cell lines, from which the normalized oxygen consumption rate (OCR) and normalized extracellular acidification rate (ECAR) were measured. The OCR was measured at baseline and in response to sequential injections of oligomycin (O), carbonyl cyanide-p-trifluoromethoxyphenyl-hydrazon (FCCP), and rotenone with antimycin (RA).

FIG. 3C depicts seahorse mitochondrial stress assay of 2 and 5-FU (positive control) toward FHC cell lines, from which the normalized oxygen consumption rate (OCR) and normalized extracellular acidification rate (ECAR) were measured. The OCR was measured at baseline and in response to sequential injections of oligomycin (O), carbonyl cyanide-p-trifluoromethoxyphenyl-hydrazon (FCCP), and rotenone with antimycin (RA).

FIG. 4A depicts Crystal violet cell viability assay of p-coumaric acid (3) and L-tyrosine (4) toward PMP, HT-29 and FHC cell lines.

FIG. 4B depicts seahorse mitochondrial stress assay of 3 and 4 toward PMP501-1 cell lines, from which the normalized oxygen consumption rate (OCR) and normalized extracellular acidification rate (ECAR) were measured. The OCR was measured at baseline and in response to sequential injections of oligomycin (O), carbonyl cyanide-p-trifluoromethoxyphenyl-hydrazon (FCCP), and rotenone with antimycin (RA).

FIG. 4C depicts seahorse mitochondrial stress assay of 3 and 4 toward ABX023-1 cell lines, from which the normalized oxygen consumption rate (OCR) and normalized extracellular acidification rate (ECAR) were measured. The OCR was measured at baseline and in response to sequential injections of oligomycin (O), carbonyl cyanide-p-trifluoromethoxyphenyl-hydrazon (FCCP), and rotenone with antimycin (RA).

FIG. 5A depicts Streptomyces sp. PMP498-3 cultivated on ISP-2 agar grows slowly and does not produce spores under aerobic condition.

FIG. 5B depicts Streptomyces sp. PMP498-3 cultivated in M9 liquid media with different supplements, showing much faster growth in the presence of 0.5% mucin (a component accumulated by PMP cells) under hypoxia (a condition associated with PMP microenvironment).

Repeat use of reference characters in the present specification and drawings is intended to represent the same or analogous features or elements of the present invention.

### DETAILED DESCRIPTION

Reference will now be made in detail to various embodiments of the presently disclosed subject matter, one or more examples of which are set forth below. Each embodiment is provided by way of explanation, not limitation, of the subject matter. In fact, it will be apparent to those skilled in the art that various modifications and variations may be made to the present disclosure without departing from the scope or spirit of the disclosure. For instance, features illustrated or described as part of one embodiment may be used in another embodiment to yield a still further embodiment. Thus, it is intended that the present disclosure covers such modifications and variations as come within the scope of the appended claims and their equivalents.

In general, disclosed herein are compositions and methods for treating pseudomyxoma peritonei. Natural products have long been considered one of the most significant sources for the discovery of bioactive small molecules in drug discovery and development. While various approaches are employed for natural product discovery, forward chemical genetic screening has proven to be the most efficient method in search for bioactive small molecules. This approach first requires the establishment of a reliable screening platform. In the case of PMP, no such screening platforms have been developed. Interestingly, the present disclosure provides stable PMP cell lines with favorable morphology for screening, directly derived from patient peritoneal tumor tissues, presents a significant advancement in PMP drug discovery. Methods disclosed herein utilizes PMP cell lines disclosed herein to screen a metabolite library to identify PMP inhibitors with potent activity against PMP.

In some example embodiments, a pseudomyxoma peritonei (PMP) inhibitor may be a myxomapeptin or an analog thereof. As used herein, “myxomapeptin” refers to a new class of depsi cyclic lipopeptides biosynthesized by myx cluster. Myxomapeptin compounds disclosed herein may have eight amino acid residues that features N-methylation, C-methylation and hydroxylation, and a-proton epimerization. In some example embodiments, myxomapeptin, or an analog thereof, may have the general structure of Formula I:

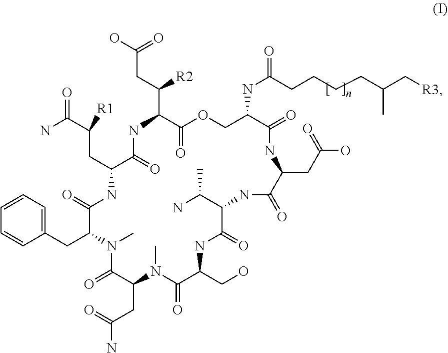

or a pharmaceutically acceptable salt thereof, wherein:

R1 through R3 may be independently selected from hydrogen,—OH, or —CH₃; and
n may be 6 or 7.

In some example embodiments, R1 may be independently selected from hydrogen,—OH, or —CH₃. For instance, in one example embodiment, R1 may be hydrogen. In another example embodiment, R1 may be—OH.

In some example embodiments, R2 may be independently selected from hydrogen,—OH, or —CH₃. For instance, in one example embodiment, R2 may be hydrogen. In another example embodiment, R2 may be—CH₃.

In some example embodiments, R3 may be independently selected from hydrogen,—OH, or —CH₃. For instance, in one example embodiment, R³ may be hydrogen. In another example embodiment, R³ may be—CH₃.

In some example embodiments, n may be 6 or 7. For instance, in one example embodiment, n may be 6. In another example embodiment, n may be 7.

In one example embodiment, the myxomapeptin of Formula I, or an analog thereof, may be represented by the following structure:

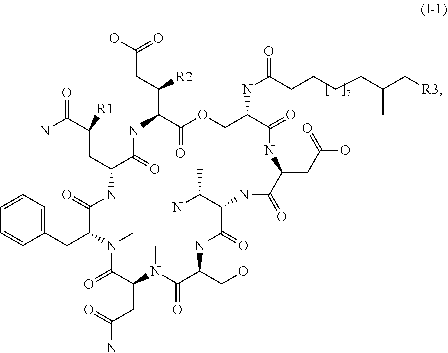

or a pharmaceutically acceptable salt thereof.

In one example embodiment, the myxomapeptin of Formula II, or an analog thereof, may be represented by the following structure:

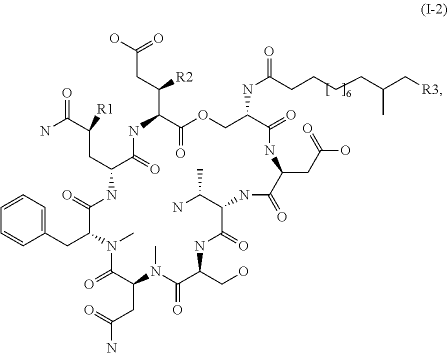

or a pharmaceutically acceptable salt thereof.

In some example embodiments, the PMP inhibitor of Formula I may be a compound having one of the following structures:

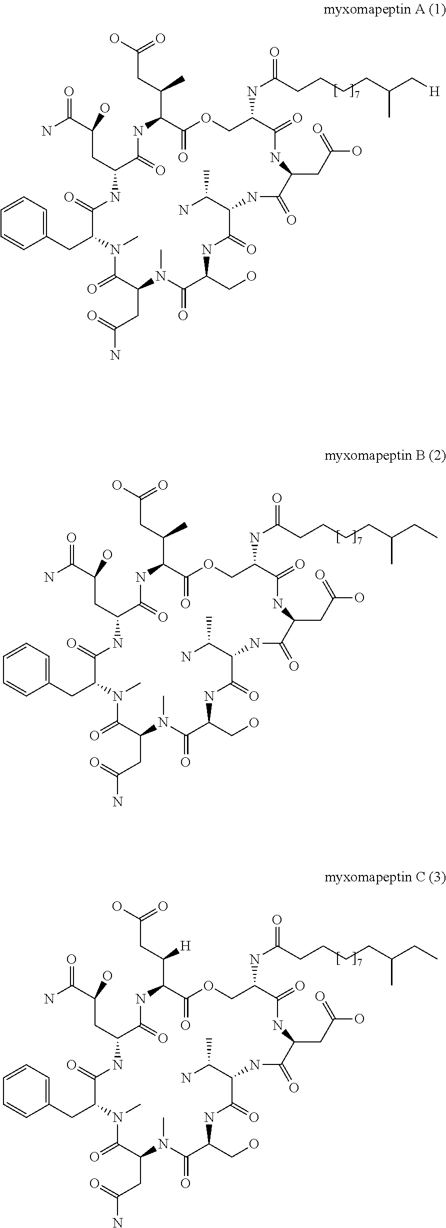

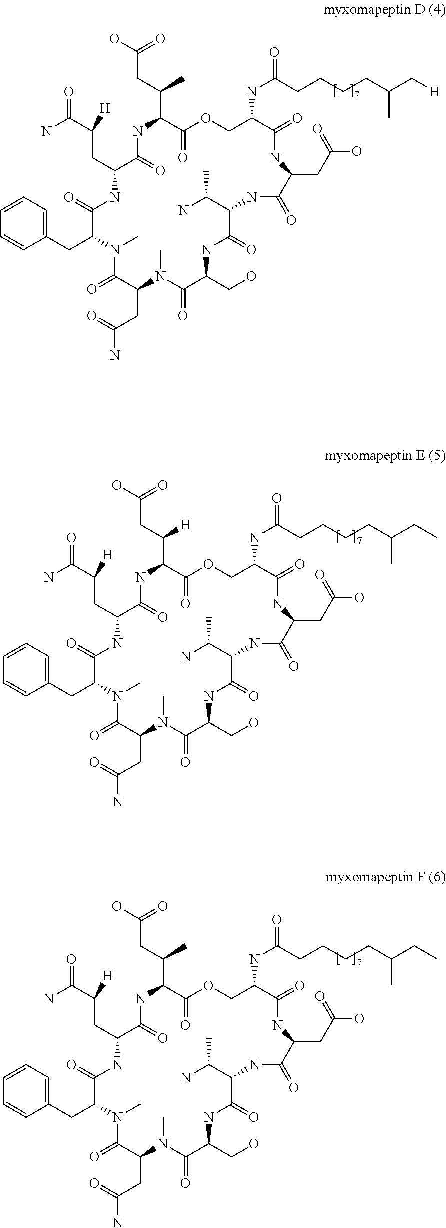

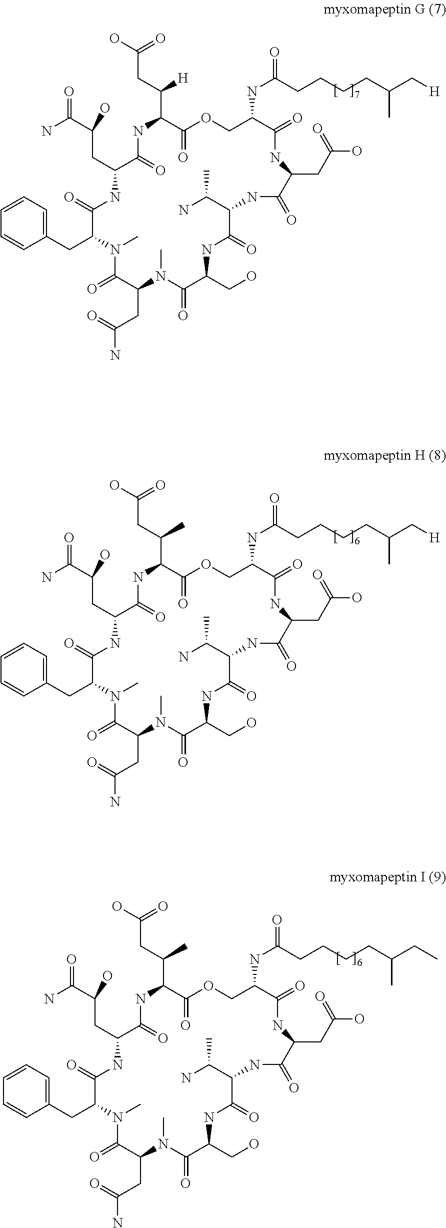

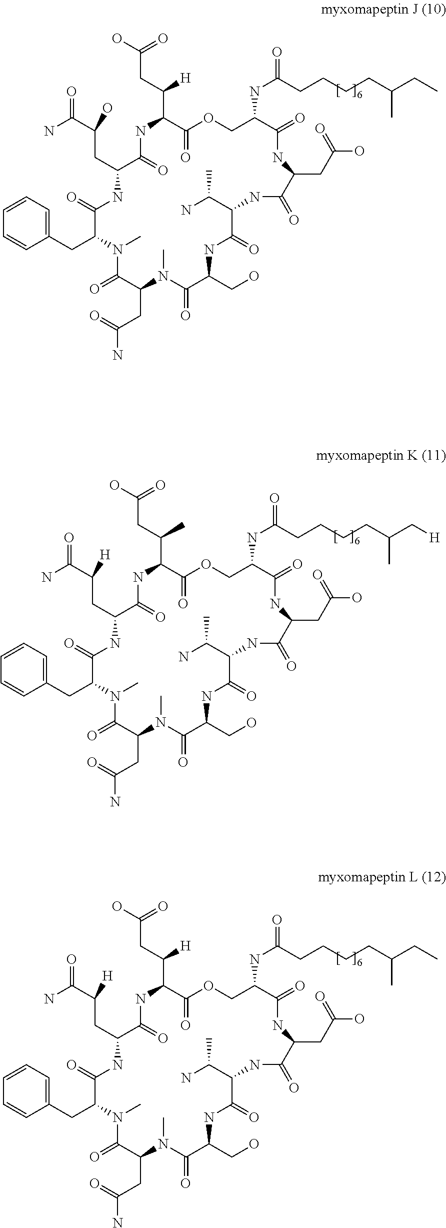

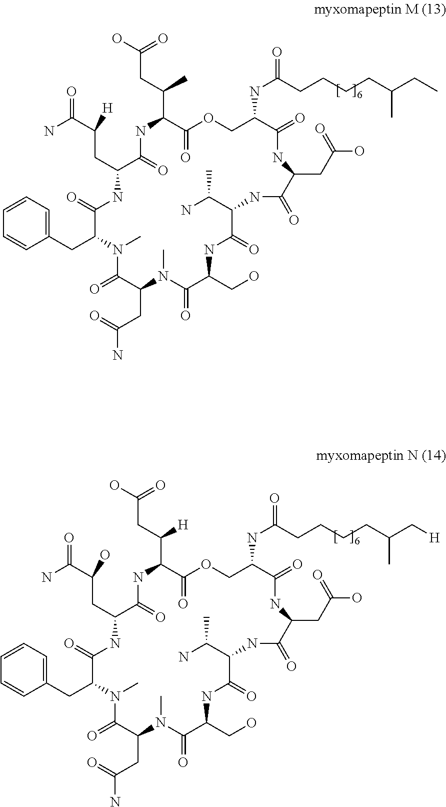

In some example embodiments, pseudomyxoma peritonei inhibitors disclosed herein may be capable of modulating activity of a metabolite expressed in peritonei cells. For instance, pseudomyxoma peritonei inhibitors disclosed herein may be capable of selectively inhibiting or decreasing pseudomyxoma peritonei activity compared to the activity of a different inhibitor. In one example embodiment, the selectivity of a pseudomyxoma peritonei inhibitor disclosed herein in inhibiting the activity of pseudomyxoma peritonei may be measured by its cytotoxicity against a pseudomyxoma peritonei cell line based on IC50 values compared to another pseudomyxoma peritonei inhibitor. It is understood that a compound with a lower IC50 value, as determined under substantially similar conditions, is a more potent inhibitor relative to a compound with a higher IC50 value. In certain embodiments, the selectivity may be at least 2-fold, at least 3-fold, at least 5-fold, at least 10-fold, at least 30-fold, at least 100-fold, at least 300-fold, at least 1,000-fold, at least 3,000-fold, at least 10,000-fold, at least 30,000-fold, or at least 100,000-fold. In some example embodiments, the selectivity may be not more than 100,000-fold, not more than 10,000-fold, not more than 1,000-fold, not more than 100-fold, not more than 10-fold, or not more than 2-fold.

In some example embodiments, pseudomyxoma peritonei inhibitors disclosed herein may be useful for treating a subject in need thereof. The term “treating” as used herein refers to partially or completely alleviating, improving, relieving, inhibiting progression, and/or reducing incidence of one or more symptoms of a disease, disorder, and/or condition, e.g., cancer.

The term “treating cancer” or “treatment of cancer” may refer to administration of a pseudomyxoma peritonei inhibitor to a subject afflicted with or at risk of a cancerous condition, and may refer to an effect that alleviates the cancerous condition by killing the cancerous cells, but also an effect that result in the inhibition of growth and/or metastasis of the cancer.

In some example embodiments, the cancer may include, but is not limited to, pseudomyxoma peritonei, mucinous ovarian cancer, appendiceal adenocarcinoma, mucinous colorectal cancer, breast cancer, colon cancer, stomach cancer, leukemia, melanoma, prostate, or renal cancer. In one example embodiment, the cancer may be pseudomyxoma peritonei. In another example embodiment, the cancer may be stomach cancer. In another example embodiment, the cancer may be mucinous ovarian cancer.

The term “subject” refers to any organism to which aspects of the disclosure can be administered, e.g., for experimental, diagnostic, prophylactic, and/or therapeutic purposes. Subjects to which embodiments of the disclosure can be administered include mammals, such as primates, for example, humans. For veterinary applications, a wide variety of subjects are suitable, e.g., livestock such as cattle, sheep, goats, cows, swine, and the like; poultry such as chickens, ducks, geese, turkeys, and the like; and domesticated animals, such as pets such as dogs and cats. In one example embodiment, the subject may include, but is not limited to, a cell, a tissue, or a mammal. For instance, a composition disclosed herein may be delivered to a cell or a tissue of the subject. For diagnostic or research applications, a wide variety of mammals are suitable subjects, including rodents (e.g., mice, rats, hamsters), rabbits, primates, and swine such as inbred pigs and the like. The term “living subject” can refer to a subject noted above or another organism that is alive. The term “living subject” can refer to the entire subject or organism and not just a part excised (e.g., a liver or other organ) from the living subject.

As used herein, the term “administration” refers to introducing a substance (e.g., a pseudomyxoma peritonei inhibitor, an exogenous antigen, a cytotoxic agent, etc.) into a subject. The administration thereof can be carried out in any convenient manner, including by aerosol inhalation, injection, ingestion, transfusion, implantation, or transplantation. For instance, the cannabinoid compound may be administered orally, subcutaneously, intravenously, or intratumoral. In this regard, “oral” administration can refer to administration into a subject's mouth; “subcutaneous” administration can refer to administration just below the skin; “intravenous” administration can refer to administration into a vein of a subject; and “intratumoral” administration can refer to administration within a tumor.

Pharmaceutical compositions disclosed herein may be formulated to be compatible with its intended route of administration. As used herein, “routes of administration” may include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EMTM (BASF, Parsippany, N.J.) or phosphate buffered saline (PBS). The composition can be sterile and should be fluid to the extent that easy syringability exists. It can be stable under the conditions of manufacture and storage and must be preserved against the contaminating action of microorganisms such as bacteria and fungi. The carrier can be a solvent or dispersion medium containing, for example, water, ethanol, a pharmaceutically acceptable polyol like glycerol, propylene glycol, liquid polyethylene glycol, and suitable mixtures thereof. Prolonged absorption of the injectable compositions may be brought about by including in the composition an agent which delays absorption, for example, aluminum monostearate and gelatin.

Oral compositions may include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier may be applied orally.

Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

Compositions for parenteral delivery, e.g., via injection, can include pharmaceutically acceptable sterile aqueous or nonaqueous solutions, dispersions, suspensions, or emulsions, as well as sterile powders for reconstitution into sterile injectable solutions or dispersions just prior to use. Examples of suitable aqueous and nonaqueous carriers, diluents, solvents or vehicles include water, ethanol, polyols (e.g., glycerol, propylene glycol, polyethylene glycol and the like), carboxymethylcellulose and suitable mixtures thereof, vegetable oils (e.g., corn oil) and injectable organic esters such as ethyl oleate. In addition, the composition can contain minor amounts of auxiliary substances such as wetting or emulsifying agents, pH buffering agents and the like that can enhance the effectiveness of the phenolic compound. Proper fluidity may be maintained, for example, by the use of coating materials such as lecithin, by the maintenance of the required particle size in the case of dispersions and by the use of surfactants. These compositions may also contain adjuvants such as preservatives, wetting agents, emulsifying agents, and dispersing agents.

In one example embodiment, a therapeutically effective amount of the pseudomyxoma peritonei inhibitor may be administered to the subject. The term “therapeutically effective amount” refers to those amounts that, when administered to a subject in view of the nature and severity of that subject's disease or condition, will have a desired therapeutic effect, e.g., an amount which will cure, prevent, inhibit, or at least partially arrest or partially prevent a target disease or condition. A therapeutically effective dose further can refer to that amount of the therapeutic agent sufficient to result in amelioration of symptoms, e.g., treatment, healing, prevention or amelioration of the relevant medical condition, or an increase in rate of treatment, healing, prevention or amelioration of such conditions. When applied to an individual active ingredient administered alone, a therapeutically effective dose can refer to that ingredient alone. When applied to a combination, a therapeutically effective dose can refer to combined amounts of the active ingredients that result in the therapeutic effect, whether administered in combination, serially or simultaneously.

A therapeutically effective dose can depend upon a number of factors known to those of ordinary skill in the art. The dosage can vary depending upon known factors such as the pharmacodynamic characteristics of the active ingredient and its mode and route of administration; time of administration of active ingredient; identity, size, condition, age, sex, health and weight of the subject or sample being treated; nature and extent of symptoms; kind of concurrent treatment, frequency of treatment and the effect desired; and rate of excretion. These amounts can be readily determined by the skilled artisan.

In one example embodiment, the therapeutically effective amount is at least about 0.1 μM body weight, at least about 0.5 μM body weight, at least about 1.0 μM body weight, at least about 1.5 μM body weight, at least about 3 μM body weight, at least about 5 μM body weight, at least about 7.5 μM body weight, at least about 10 μM body weight, at least about 15 μM body weight, at least about 20 μM body weight, at least about 25 μM body weight, at least about 30 μM body weight, at least about 35 μM body weight, at least about 40 μM body weight, at least about 50 μM body weight.

In some example embodiments, for instance, the pseudomyxoma peritonei inhibitor may be administered to a subject at a dosage of from about 0.1 μM body weight to about 50 μM body weight, such as from about 1 μM body weight to about 25 μM body weight, such as from about 5 μM body weight to about 20 μM body weight, or any range therebetween.

Pharmaceutical compositions as described herein can be administered to the subject one time (e.g., as a single injection or deposition). Alternatively, administration can be once or twice daily to a subject in need thereof for a period of from about 2 days to about 35 days, such as from about 7 days to about 28 days, such as from about 10 to about 21 days, or any range therebetween. It can also be administered once or twice daily to a subject for a period of 1, 2, 3, 4, 5, 6, 7, 8, 9, 10, 11, 12 times per year, or a combination thereof.

The present disclosure provides methods for identifying and/or screening inhibitors that may be useful for the treatment and/or prevention of pseudomyxoma peritonei. In some embodiments, methods for identifying and/or screening inhibitors against pseudomyxoma peritonei may include establishing a stable cell line. As used herein, a “cell line” may refer to cells that are cultured in vitro. For instance, the cells may include a myoblast, a fibroblast, a glioblastoma, a carcinoma, an epithelial cell, an immune cell, a stromal cell, a mesothelial, or a stem cell. In one example embodiment, the cells may be immune cells, such as T cell, B cell, macrophage, a natural killer (NK) cell or dendritic cell.

In one example embodiment, the cell may be a primary cell. For instance, the primary cell may be a neuron, a cardiomyocyte or a primary immune cell. As used herein, “primary” in the context of a primary cell refers to a cell that has not been transformed or immortalized. Such primary cells can be cultured, sub-cultured, or passaged a limited number of times. Primary cells, for instance, may be isolated from an organism, system, organ, or tissue, optionally sorted, and utilized directly without culturing or sub-culturing.

In one example embodiment, the cell line may be established from a peritoneal cell. As used herein, “peritoneal cells” may refer to the cells that make up the peritoneum, which is a thin, double-layered serous membrane that lines the abdominal cavity and covers the abdominal organs. In some example embodiments, a peritoneal cell line may include, but is not limited to, PMP498-1, PMP501-1, or ABX023. In one example embodiment, the peritoneal cell line may be PMP498-1. In another example embodiment, the peritoneal cell line may be PMP501-1. In yet another example embodiment, the peritoneal cell line may be ABX023.

In some example embodiments, inhibitors that may be useful for the treatment and/or prevention of pseudomyxoma peritonei may be screened against a peritoneal cell line. For instance, the activity or level of a pseudomyxoma peritonei inhibitor may be detected and/or quantified by measuring a level of one or more metabolites selected from Table 1 in a peritoneal cell line. Subsequently, expression levels of one or more metabolites selected from Table 1 may be compared to the level of the one or more metabolites in the cell line before contact with a PMP inhibitor. In one example embodiment, the presence of, or a significant increase in, the expression level of metabolite after contacting the stable cell line with the inhibitor indicates that the inhibitor effectively treats pseudomyxoma peritonei.

The pseudomyxoma peritonei inhibitor or metabolite may be detected and/or quantified by any of a number of means well-known to those of skill in the art. Any method known in the art for detecting inhibitors may be used. Such methods include, but are not limited to, immunodiffusion, immunoelectrophoresis, radioimmunoassay (RIA), enzyme-linked immunosorbent assays (ELISAs), immunofluorescent assays, Western blotting, binder-ligand assays, immunohistochemical techniques, agglutination, complement assays, high performance liquid chromatography (HPLC), thin layer chromatography (TLC), hyperdiffusion chromatography, or a combination thereof.

The preceding description is exemplary in nature and is not intended to limit the scope, applicability or configuration of the disclosure in any way. Various changes to the described embodiments may be made in the function and arrangement of the elements described herein without departing from the scope of the disclosure.

Here and throughout the specification and claims, range limitations are combined and interchanged, such ranges are identified and include all the sub-ranges contained therein unless context or language indicates otherwise. For example, all ranges disclosed herein are inclusive of the endpoints, and the endpoints are independently combinable with each other.

This written description uses examples to disclose the present disclosure, including the best mode, and also to enable any person skilled in the art to practice the disclosure, including making and using any devices or systems and performing any incorporated methods. The patentable scope of the disclosure is defined by the claims, and may include other examples that occur to those skilled in the art. Such other examples are intended to be within the scope of the claims if they include structural elements that do not differ from the literal language of the claims, or if they include equivalent structural elements with insubstantial differences from the literal languages of the claims.

It is to be understood that the terminology used herein is for the purpose of describing particular embodiments only and is not intended to limit the scope of the present disclosure. In this specification and in the claims that follow, reference will be made to a number of terms that shall be defined to have the following meanings.

Unless defined otherwise, all technical and scientific terms used herein have the same meaning as commonly understood by one of ordinary skill in the art to which this invention is related.

As used in this application and in the claims, the singular forms “a”, “an”, and “the” include the plural forms unless the context clearly dictates otherwise. Additionally, the term “includes” means “comprises”. The methods and compositions of the present disclosure, including components thereof, can comprise, consist of, or consist essentially of the essential elements and limitations of the embodiments described herein, as well as any additional or optional ingredients, components or limitations described herein or otherwise useful in biocidal compositions.

Unless otherwise indicated, all numbers expressing quantities of ingredients, properties such as molecular weight, percentages, and so forth, as used in the specification or claims are to be understood as being modified by the term “about”. Accordingly, unless otherwise indicated, implicitly or explicitly, the numerical parameters set forth are approximations that may depend on the desired properties sought and/or limits of detection under standard test conditions/methods. When directly and explicitly distinguishing embodiments from discussed prior art, the embodiment numbers are not approximate unless the word “about” is recited.

As used herein, “optional” or “optionally” means that the subsequently described material, event or circumstance may or may not be present or occur, and that the description includes instances where the material, event or circumstance is present or occurs and instances in which it does not. As used herein, “w/w %” and “wt %” mean by weight as relative to another component or a percentage of the total weight in the composition.

The term “about” is intended to mean approximately, in the region of, roughly, or around. When the term “about” is used in conjunction with a numerical range, it modifies that range by extending the boundaries above and below the numerical values set forth. Unless otherwise indicated, it should be understood that the numerical parameters set forth in the following specification and attached claims are approximations. At the very least, and not as an attempt to limit the application of the doctrine of equivalents to the scope of the claims, numerical parameters should be read in light of the number of reported significant digits and the application of ordinary rounding techniques.

The phrase “effective amount” means an amount of a compound that promotes, improves, stimulates, or encourages a response to the particular condition or disorder or the particular symptom of the condition or disorder.

Furthermore, certain aspects of the present disclosure may be better understood according to the following examples, which are intended to be non-limiting and exemplary in nature. Moreover, it will be understood that the compositions described in the examples may be substantially free of any substance not expressly described.

The present disclosure may be better understood with reference to the following examples.

### EXAMPLES

### Example 1

Prioritization of PMP-associated bacteria that modulate PMP disease

Using a 16S amplicon-based sequence analysis, we have identified a core microbiome comprising of 32 genera from PMP tumor and mucin tissues collected aseptically from 11 patients. Through cultivating these tissue samples under microaerophilic conditions, 40 bacterial strains were isolated that all belong to the PMP core microbiome (Table 1). To prioritize the PMP-associated bacteria that exert the greatest impact on the growth and proliferation of PMP tumors, bacterial isolates that (i) have unique taxonomic traits, (ii) can penetrate PMP tumor cells, and (iii) inhibit or promote PMP cell proliferation specifically rather than other tumor cells (e.g. HT-29) were prioritized for chemical and biological studies. Following the criteria above, two PMP-associated strains PMP498-3 and PMP191F were prioritized for study, as described below.

TABLE 1
PMP-associated bacteria isolated.

| No. | Designation | Taxonomy |
|---|---|---|
| 1 | PMP191C | Dermacoccus (genus) |
| 2 | PMP191M | Bosea (genus) |
| 3 | PMP191F | Chitonophagaceae (family) |
| 4 | PMP213A | Cutibacterium acnes (genus) |
| 5 | PMP213BC | Cutibacterium acnes (genus) |
| 6 | PMP215 | Amycolatopsis (genus) |
| 7 | PMP238 | Corynebacterium (genus) |
| 8 | PMP498-3 | Streptomyces (genus) |
| 9 | PMP196 | Cutibacterium acnes (genus) |
| 10 | PMP219 | Cutibacterium acnes (genus) |
| 11 | PMP229 | Cutibacterium acnes (genus) |
| 12 | PMP267-3 | Cutibacterium acnes (genus) |
| 13 | PMP267B | Corynebacterium (genus) |
| 14 | PMP274 | Cutibacterium acnes (genus) |
| 15 | PMP285 | Cutibacterium acnes (genus) |
| 16 | PMP324 | Cutibacterium acnes (genus) |
| 17 | PMP343 | Streptococcus anginosus (species) |
| 18 | PMP350 | Enterococcus (genus) |
| 19 | PMP352A | Enterococcus (genus) |
| 20 | PMP352B | Enterococcus faecalis (species) |
| 21 | PMP354 | Propionibacterium (genus) |
| 22 | PMP374W | Staphylococcus warneri (species) |
| 23 | PMP374S | Micrococcus (genus) |
| 24 | PMP374Y | Micrococcus (genus) |
| 25 | PMP378B | Micrococcus (genus) |
| 26 | PMP378-5 | Escherichia coli (species) |
| 27 | PMP376-5 | Escherichia coli (species) |
| 28 | PMP395 | Corynebacterium (genus) |
| 29 | PMP433 | Streptococcus mitis (species) |
| 30 | PMP439 | Pantoea ananatis (species) |
| 31 | PMP441-3 | Stenotrophomonas maltophila (species) |
| 32 | PMP454-4 | Stenotrophomonas maltophila (species) |
| 33 | PMP459-2 | Enterococcus faecium (species) |
| 34 | PMP454-5 | Stenotrophomonas maltophila (species) |
| 35 | PMP454-7 | Stenotrophomonas maltophila (species) |
| 36 | PMP471-6 | Staphylococcus epidermidis (species) |
| 37 | PMP471-7 | Staphylococcus epidermidis (species) |
| 38 | C-07-1 | Actinomyces turicensis (species) |
| 39 | C-07-2 | Actinomyces europaeus (species) |
| 40 | C-07-3 | Actinomyces europaeus (species) |

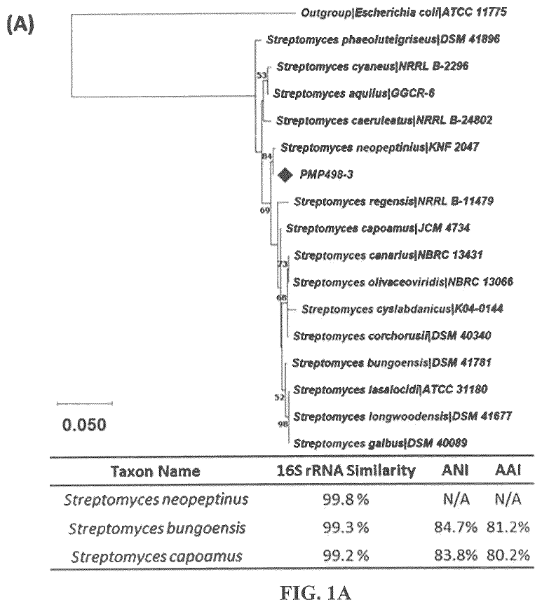

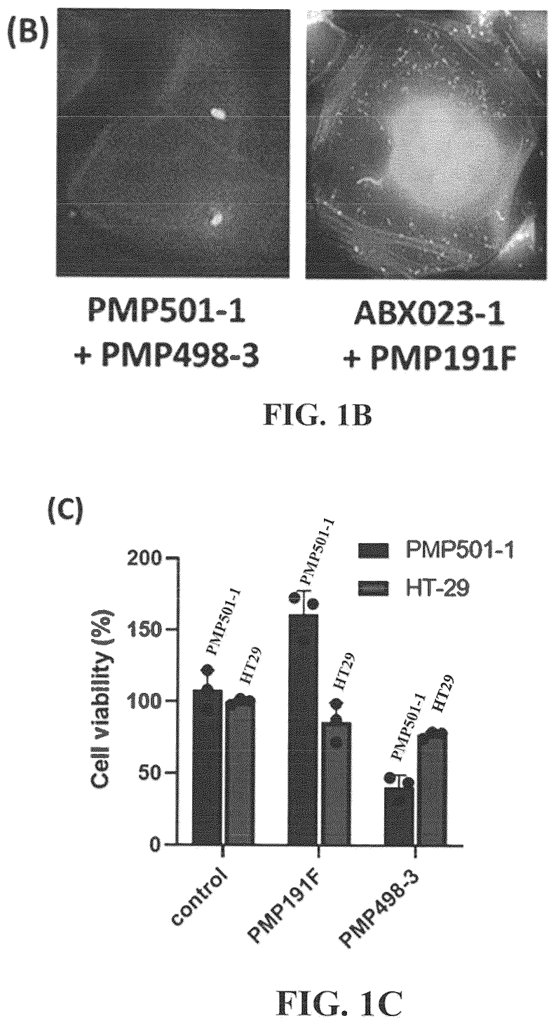

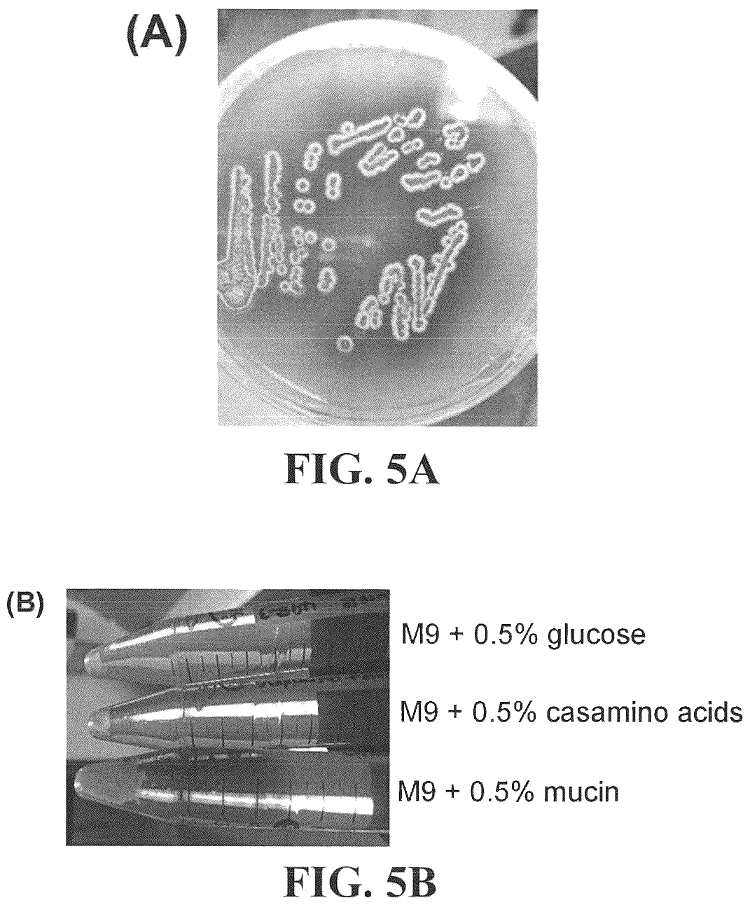

16S rRNA gene analysis revealed that PMP498-3 belongs to the genus Streptomyces as it shares high sequence similarity with Streptomyces neopeptinus (99.8%; EU258679), Streptomyces bungoensis (99.3%; KQ948892), and Streptomyces capoamus (99.2%; AB045877) in BLAST search (FIG. 1A). However, when the genome of PMP498-3 was compared to those of S. bungoensis and S. capoamus (S. neopeptinus cannot be compared due to the unavailability of its genome), the average nucleotide identity (ANI) values were 84.7% and 83.8%, respectively, much lower than the 95% cutoff value for species identification, suggesting that PMP498-3 is a new Streptomyces species (FIG. 1A). PMP498-3 grows slowly and does not produce spores on agar plates under aerobic condition (FIG. 5A). Interestingly, its growth becomes faster in the presence of 0.5% mucin (a component accumulated by PMP cells) under hypoxia (a condition associated with PMP microenvironment) in liquid media (FIG. 5B). Similarly, according to the 16S rRNA gene and genome-wide phylogenetic analyses, PMP191F was classified as a member of the Chitinophagaceae family and assigned to an undefined genus, which we have newly designated as Parapseudoflavitalea muciniphila.

Next, whether PMP-associated bacteria can enter PMP tumor cells was evaluated using fluorescence imaging assay. To this end, PMP cells and the bacteria stained by carboxyfluorescein diacetate succinimidyl ester (CFDA-SE) were co-incubated for 3 hours. Using a Keyence fluorescence microscope, we observed the presence of PMP498-3 and PMP191F bacterial cells within the cytoplasma of PMP501-1 tumor cells (FIG. 1B). This observation provided evidence that PMP498-3 and PMP191F are capable of penetrating PMP501-1 tumor cells and suggested a close interaction with PMP cells.

In cell viability assay, at a concentration of 50 mg/mL, the butanol extract of PMP498-3 inhibited the proliferation of PMP501-1 by approximately 55%, but had a limited effect on the human colorectal cancer cell line HT-29 (FIG. 1C). In contrast, the butanol extract of PMP191F enhances the growth of PMP501-1 by approximately 50% but did not exhibit any detectable effect on HT-29 (FIG. 1C). These results suggest that these strains highly likely produce small molecules that modulate PMP cells.

Based on their distinct taxonomic characteristics, close interaction with PMP cells, and specific activities towards PMP cells, without wishing to be bound by theory, it was proposed that PMP498-3 and PMP191F may play a potential role in modulating the progression of PMP diseases through their secondary metabolites. This prompted us to explore the underlying chemical logic from the two candidate PMP-associated strains.

### Example 2

Bioassay-guided fractionation of PMP498-3 led to the discovery of a new class of lipopeptides

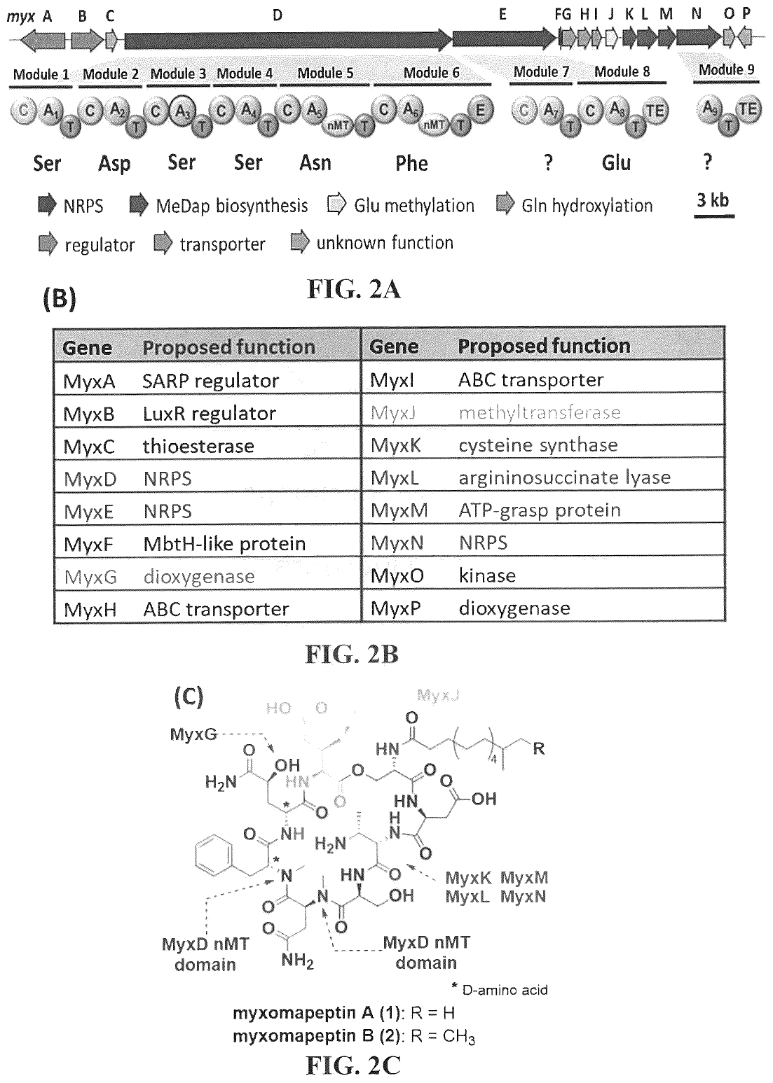

To investigate the metabolites produced by Streptomyces sp. PMP498-3 that inhibit PMP cell proliferation, we cultivated the strain in 12 L ISP-2 broth supplemented with 0.5% mucin followed by butanol extraction. Guided by cell viability assay, the extract was fractionated using reverse phase C18 and Sephadex LH-20 columns leading to the active fraction containing a series of peaks with m/z ranging from 1160-1250 Da. Two major compounds 1 and 2 were further purified from the active fraction. High-resolution mass spectrometry (HR-MS) analysis of 1 and 2 returned protonated ions consistent with the molecular formulas C54H85N11018 (Ammu-0.8) and C55H87N11018 (Ammu-0.4), respectively. One-dimensional (1D) nuclear magnetic resonance (NMR) analysis of both compounds revealed diagnostic features for peptide skeleton, such as a series of resonances for a-proton (8H 4.5-5.5 ppm) and amide carbonyls (dc 170-180 ppm) of amino acids (Figures S10, 11, 18 and 19). Interestingly, there are two respective sets of NMR signals with a ratio of 3:2 for both 1 and 2 (Table S6), likely due to the presence of two major conformers in methanol-d4. Interpretation of the 1D and 2D NMR spectra permitted the identification of eight amino acid residues, including two serines (Ser), an aspartic acid (Asp), a N-methyl-phenylalanine (N-Me-Phe), a N-methyl-asparagine (N-Me-Asn), a y-hydroxy-glutamine (y-OH-Gln), a β-methyl-glutamic acid (B-Me-Glu), and a 2,3-diaminobutanoic acid (MeDap) in the structures of 1 and 2 (FIG. 2C). The only difference between the two structures is the length of the lipid tail connected to Ser. The connectivity of each amino acid residue was subsequently established based on the HMBC and ROESY NMR correlations together with the bioinformatics-based domain prediction (FIG. 2A). The peptide chain is proved to be cyclized through the ester bond between Ser1 and B-Me-Glu8 and the lipid tail anchors to Ser1 through an amide group by 2D NMR analysis. Because 1 and 2 were difficult to be fragmented by tandem high-resolution mass spectrometry (HR-MS/MS), we thus linearized 1 and 2 by hydrolysing the ester bond in a mild alkaline condition. HR-MS/MS analysis of the linearized 1 and 2 give a set of b ions (fragment ions retaining N-terminus) and y ions (fragment ions retaining C-terminus), which allowed the elaboration of the sequences of 1 and 2.

Next, the absolute configuration of amino acid residues in 1 and 2 were determined by advanced Marfey's analysis after acid hydrolysis. After derivatization with (L)-and (D)-1-fluoro-2-4-dinitrophenyl-5-L-alanine amide (FDAA), the acid hydrolysate of 1 and 2 together with amino acid standards were analyzed by LCMS. Results revealed that N-Me-Phe6 and y-OH-Gln7 are D-amino acids, whereas other residues are in L-configuration. Besides, the analysis allowed assignment of non-canonical amino acids as (2S,3R)-MeDap, (2R,4S)—Y—OH-Gln and (2S,3R)-B-Me-Glu in the structures of 1 and 2. Thus, the structures inclusive of absolute configuration of 1 and 2 are unambiguously assigned (FIG. 2C), and were named myxomapeptins A and B based on the source of the producing strain. Myxomapeptins are a new class of depsi cyclic lipopeptide consisting of eight amino acid residues that features N-methylation, C-methylation and hydroxylation, and a-proton epimerization. Structurally, myxomapeptin is the most comparable to lipopeptin A and neopeptin, which are isolated from soil-borne Streptomyces strains, as all of them possess the same size of macrocyclic ring including N-Me-Phe and N-Me-Asn. Lipopeptin A and neopeptin were reported to possess antibacterial and antifungal activities. However, 1 and 2 have no antimicrobial activity at the concentration of 64 μg/mL, which is likely due to the fact that different stereochemistry of amino acids in myxomapeptin from those in lipopeptin A and neopeptin (all L-amino acids). Without wishing to be bound by theory, this suggested that myxomapeptin has evolved independently from lipopeptin A and neopeptin, forming a novel class of cyclic depsi-lipopeptides.

### Example 3

Biosynthesis of myxomapeptins

To elucidate the gene clusters (myx) that responsible for myxomapeptin biosynthesis, we sequenced the whole genome of Streptomyces sp. PMP498-3 using both Illumina MiSeq and PacBio sequencing technologies followed by de novo SPAdes assembly. A total of 33 putative biosynthetic gene clusters (BGCs) including 5 BGCs containing non-ribosomal peptide synthetases (NRPSs) was predicted by antiSMASH 6.0. Without wishing to bound by theory, it was postulated that nitrogen methyltransferases (nMT) domains should be present in at least two NRPS modules because 1 and 2 are NRPS origins and process two N-methylated amino acids in their structures. Using the nMT domain sequence as a probe, it was discovered that only one NRPS (myxD, ˜23 kb) has two nMT domains, and the modules in which nMTs located are predicted to incorporate N-Me-Asn and N-Me-Phe into the peptide chain (FIG. 2A), which is consistent with the structures of 1 and 2. A small NRPS gene (myxE, 2.4 kb) encoding two modules and a thioesterase domain is located downstream of myxD. The two NRPSs code for eight modules in total, which correspond to the size of myxomapeptin.

Apart from the NRPSs, the putative myx cluster also encodes SARP and LuxR regulators (MyxA and MyxB), a standalone thioesterase (MyxC), a MbtH-like protein (MyxF) that essential for NRPS function, a dioxygenase (MyxG) involved in hydroxylation of Gln, two ATP-binding cassette (ABC) transporters (MyxH and MyxI), a methyltransferase (MyxJ) for the formation of β-Me-Glu, and a MeDap biosynthesis cassette comprising of a cysteine synthase (MyxK), an argininosuccinate lyase (MyxL), an ATP-grasp protein (MyxM), and a condensation (C) domain-free NRPS (MyxN) (FIG. 2B). It is noted that the four-gene MeDap biosynthesis cassette has also been found in the BGCs of calcium-dependent lipopeptides laspartomycin and friulimicin A. Without wishing to be bound by theory, it was proposed that MyxKLM synthesizes MeDap, which is then activated and release by MyxN. The activated MeDap is subsequently incorporated into the lipid-Ser-Asp peptide chain through peptide bond formation catalyzed by the condensation domain in MyxD Module 3. Although predicted to incorporate Ser, the adenylation (A) domain in MyxD Module 3 is highly likely malfunctional and its function is replaced by MyxN A domain. Further examination of the NRPSs revealed the existence of an epimerization (E) domain in MyxD Module 6, implying that N-Me-Phe6 is in D-configuration, which is compatible with our chemical analysis. Interestingly, γ—OH-Gln7 in 1 and 2 was in D configuration as validated by Marfey's analysis, while E domain is absent in MyxE Module 7, suggesting the epimerization of γ—OH-Gln7 occurs prior to loading onto MyxE and the reaction might be catalyzed by an unknown standalone epimerase. Like most bacterial lipopeptides, the activated lipid tails stemmed from primary metabolism are introduced directly to MyxD Module 1 (C-A-T module) at the start of the biosynthesis.

To experimentally validate the involvement of myx cluster in myxomapeptin biosynthesis, we knocked out the partial region of myxD and the entire myxE using double crossover recombination. Comparative LCMS analysis of the PMP498-3 mutant and the wild-type strain revealed abolished production of 1 and 2, demonstrating myx cluster is responsible for myxomapeptin biosynthesis.

### Example 4

Myxomapeptins are specific and negative modulators of PMP cells

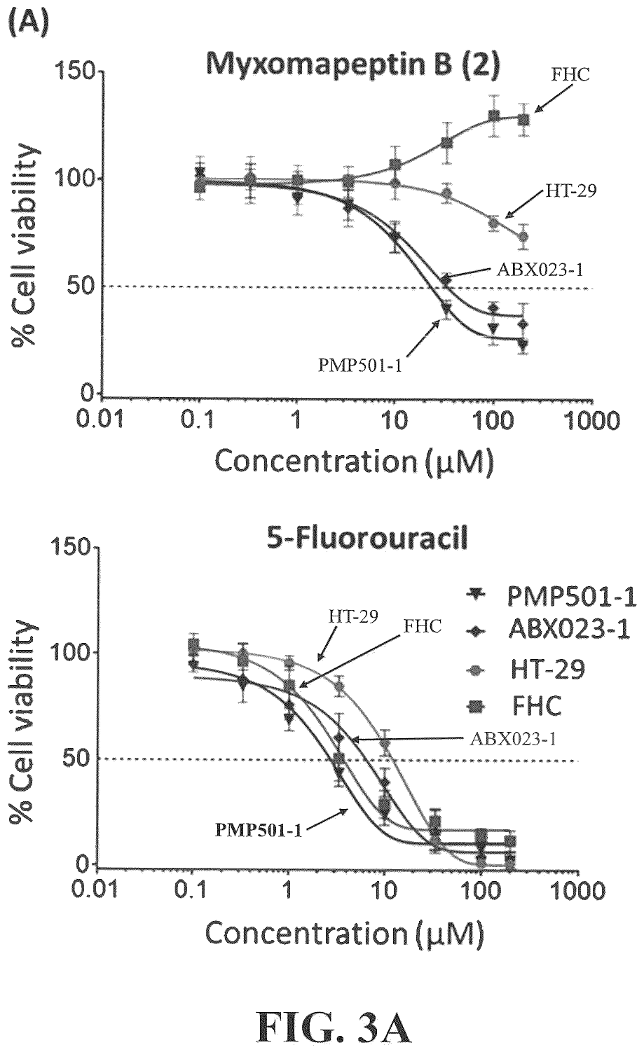

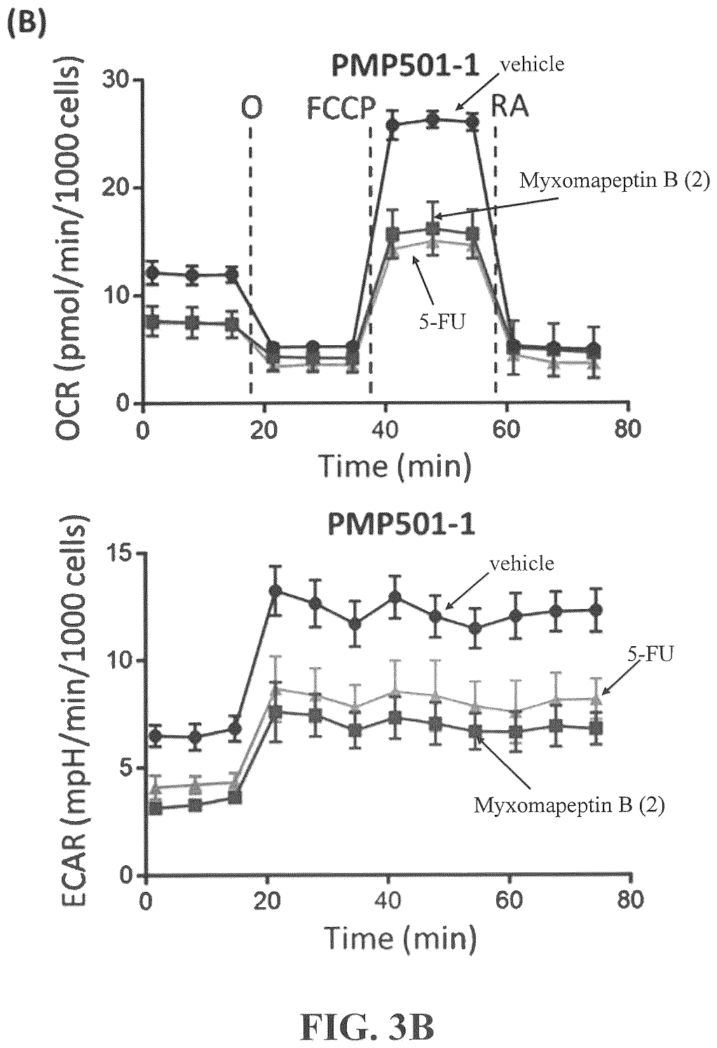

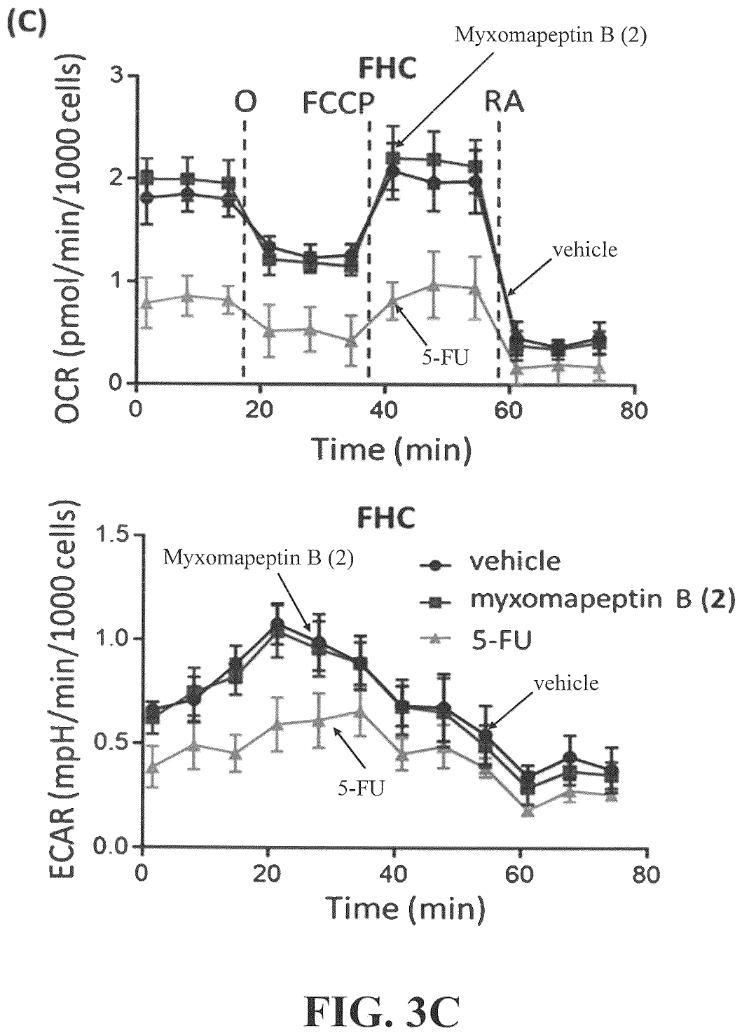

Preliminary screening of PMP498-3 butanol extract displayed inhibitory activity toward PMP501-1 cells. To evaluate whether myxomapeptin is the major contributor to the PMP inhibition, 2 were assayed against two PMP cell lines (PMP501-1 and ABX023-1), human colorectal adenocarcinoma cells (HT-29), and human fetal intestinal epithelial cells (FHC) using crystal violet cell viability assay. Consistent with the results for extract, 2 shows considerable inhibitory activity specifically against PMP cells, with IC₅₀ values of 23.0 μM for PMP501-1 and 33.7 μM for ABX023-1, while having undetectable effect on HT-29 and FHC cells (IC₅₀>100 μM) (FIG. 3A).

As tumor cells requires larger energy supply that relies on mitochondrial respiration and glycolysis, we sought to explore the effect of myxomapeptins on PMP cell energy metabolism using seahorse mitochondrial stress assay. The normalized oxygen consumption rate (OCR; proportional to mitochondrial respiration) and normalized extracellular acidification rate (ECAR; proportional to glycolysis) were measured for PMP501-1 cells and FHC in the presence of 2, 5-fluorouracil (5-FU) or vehicle. Results revealed that the basal respiration, ATP-linked respiration, and maximum respiratory capacity of PMP cells are expectedly 5-to10-fold higher compared to those for FHC cells due to the latter being normal cells (FIGS. 3B-3C). Comparable to 5-FU, 2 strongly inhibits PMP501-1 mitochondrial respiration as evidenced by significantly reduced OCR and ECAR (normalized to per 1,000 cells to eliminate the effect caused by cell cytotoxicity). Particularly, such effect of 2 was not observed for FHC cells (FIG. 3C), indicating the specific activity of 2 against the PMP cells while 5-FU unselectively inhibited the function of both PMP and FHC cells.

Both the cell viability assay and seahorse mitochondrial stress assay indicated that myxomapeptins exhibited a specific inhibitory effect on PMP cells, most likely through suppressing mitochondrial respiration and glycolysis. The moderate inhibition caused by myxomapeptin can suppress the growth and proliferation of PMP cells while avoiding a strong lethal effect, which permits both the bacteria and the PMP cells to survive.

### Example 5

PMP cells uptake myxomapeptins actively

Due to the inability of entering target cells passively, most cyclic lipopeptides are reported to perform biological functions by disrupting cell wall biosynthesis or the integrity of cell membranes. Given that myxomapeptins influence mitochondrial respiration and glycolysis, which occur in the cytoplasm, without wishing to be bound by theory, it was hypothesized that myxomapeptins are likely able to enter PMP cells. To this end, 2 was fed into the cultures of PMP501-1, ABX023-1, HT-29 and FHC cells with normalized cell density. After 5 days of incubation and a thorough wash, the cells were lysed and extracted by butanol for LCMS analysis. By calculating the ratio of 2 in cell lysis versus control extracts, 18.9% and 26.7% of 2 were found in the lysates of PMP501-1 and ABX023-1, whereas only 6.1% and 4.1% were detected from the lysates of HT-29 and FHC, respectively (FIG. 3A). In contrast, daptomycin, a cell membrane-bound antibiotic that also belongs to lipopeptides like 2, cannot be detected in the lysate of any cell lines tested after feeding. The findings suggested that myxomapeptins could be recognized and taken up by PMP cells actively, implying the specific relationship between myxomapeptins and PMP cells.

Next, to study the percentage of myxomapeptins contributed by the intracellular PMP498-3 in light of the presence of PMP498-3 bacteria inside the PMP cells, the bacteria was co-cultured with the cell lines tested above. LCMS analysis revealed 22.5% and 30.2% of 2 were found in the cell lysates of PMP501-1 and ABX023-1 (FIG. 3A), indicating the intracellular PMP498-3 contributes a small portion of myxomapeptin found inside the cells.

### Example 6

p-Coumaric acid from PMP191F is a positive modulator of PMP cells

As the extract of PMP191F enhanced the proliferation of PMP cell lines, the active components from the culture of PMP191F were purified and characterized. Bioassay-guided fractionation of 10 L culture of PMP191F led to the purification of p-coumaric acid (3), the structures of which were characterized by NMR analysis. 3 is widely distributed in microorganisms and plants and possesses bactericidal and antioxidant properties. Moreover, it was recently discovered that 3 has a variety of regulatory roles in biological situations, such as inducing oxidative stress and siderophore production in marine bacterial symbiont Phaeobacter inhibens and improving hypothalamic leptin signalling and glucose homeostasis in mice. It is reported that 3 is transformed from L-tyrosine (4) through non-oxidative deamination catalyzed by tyrosine ammonia-lyase (TAL) in bacteria. Using bacterial TAL sequences as the query, we found a TAL homologue ctg49_22 in the genome of PMP191F. BLASTP search of ctg49_22 resulted in the closest protein aromatic amino acid ammonia-lyase from Terrimonas sp.

(WP_116876142.1) with the identity of 74.3% (Figure). Thus, ctg49_22 is highly likely encodes an aromatic amino acid ammonia-lyase and responsible for the conversion of 4 to 3 in PMP191F. To verify the capability of PMP191F in converting 4 to 3, we added 4 (30 mM) to the culture of PMP191F followed by three days' cultivation. As expected, a significantly increased amount of 3 was detected, demonstrating that 3 is biosynthesized from L-tyrosine by deamination.

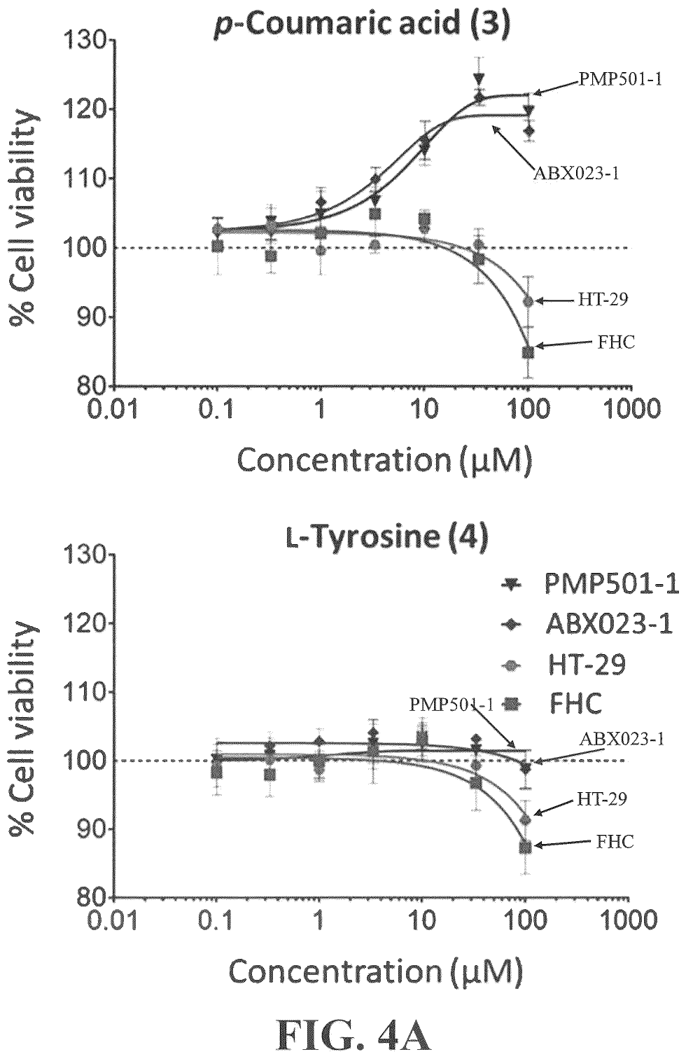

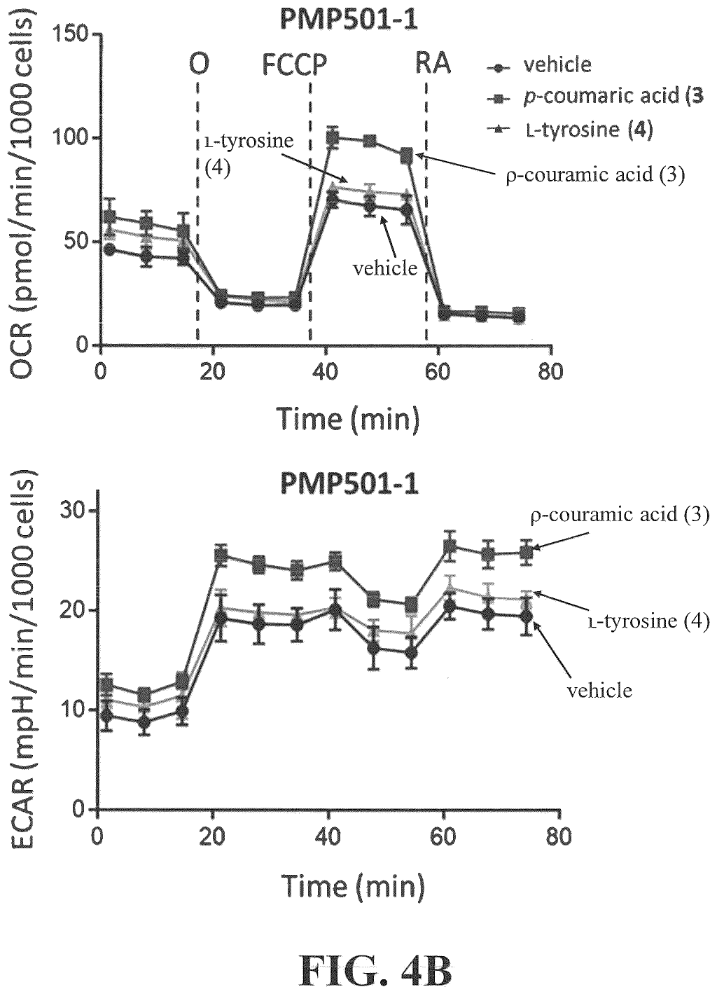

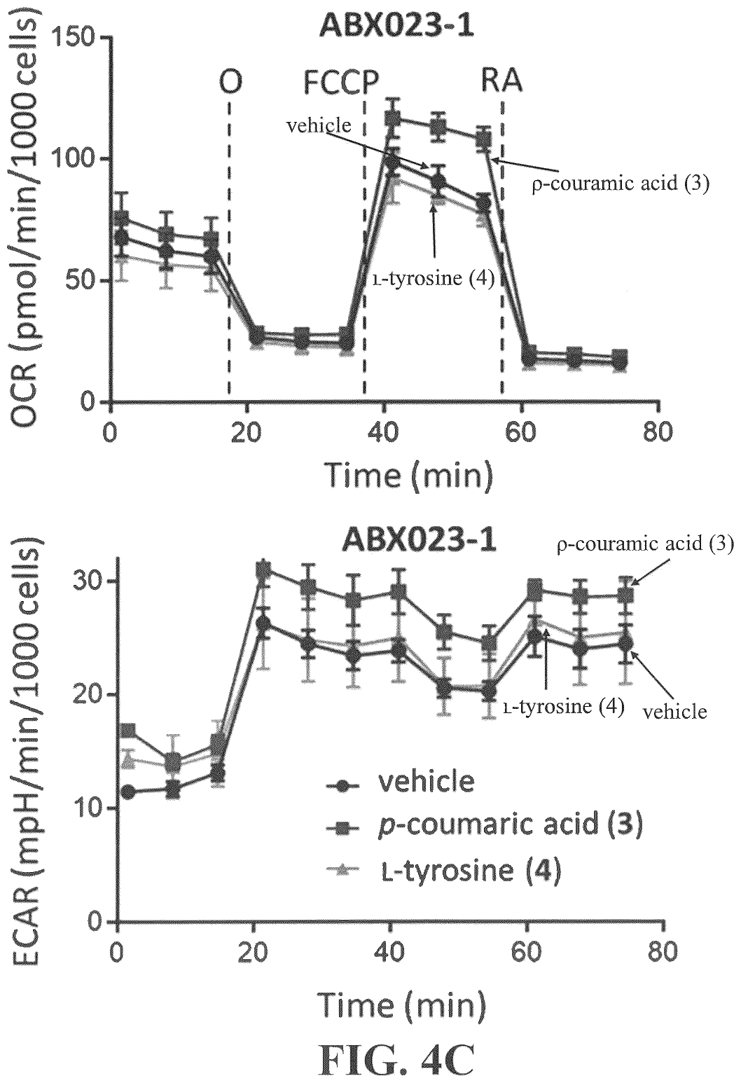

Next, the modulatory effect of 3 and 4 on PMP, HT-29 and FHC cells was evaluated. The cell viability assay demonstrated that 3 stimulates the proliferation of both PMP501-1 and ABX023-1 cells by ˜20% at the concentration of 33.3 μM, but has no effect on HT-29 or FHC cells. The amino acid 4 has no discernible effect on any of the cells tested. Subsequently, the effect of 3 and 4 on the mitochondrial respiration of ABX023-1 cells was measure in the seahorse mitochondrial stress assay. The results showed that the maximum respiratory capacity of ABX023-1 was increased by 25% in the presence of 15 μM of 3 after FCCP supplementation, while 4 do not have any effect, indicating that 3 promotes the oxygen consumption of ABX023-1. In agreement with the cell viability assay, 3 had no substantial stimulatory effect on the oxygen consumption of HT-29 and FHC cells (FIG. 4A), demonstrating that the stimulatory effect of 3 is specific to PMP cells.

These and other modifications and variations to the present invention may be practiced by those of ordinary skill in the art, without departing from the spirit and scope of the present invention, which is more particularly set forth in the appended claims. In addition, it should be understood that aspects of the various embodiments may be interchanged both in whole or in part. Furthermore, those of ordinary skill in the art will appreciate that the foregoing description is by way of example only and is not intended to limit the invention so further described in such appended claims.

What is claimed:

1. A pseudomyxoma peritonei (PMP) inhibitor of Formula I: 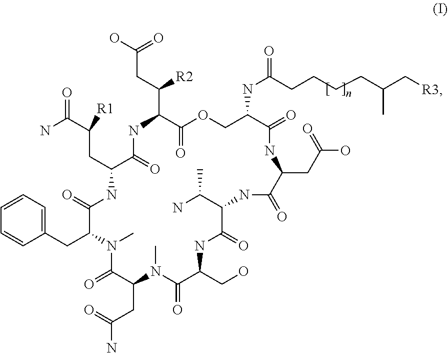
or a pharmaceutically acceptable salt thereof, wherein:
R1 through R3 is independently selected from hydrogen,—OH, or —CH₃; and
n is 6 or 7.

2. The PMP inhibitor of claim 1, wherein R1 is hydrogen or —OH.

3. The PMP inhibitor of claim 1, wherein R2 or R3 is independently selected from hydrogen or —CH₃.

4. The PMP inhibitor of claim 1, wherein n is 6.

5. The PMP inhibitor of claim 1, wherein n is 7.

6. The PMP inhibitor of claim 1, wherein the PMP inhibitor has the following structure: 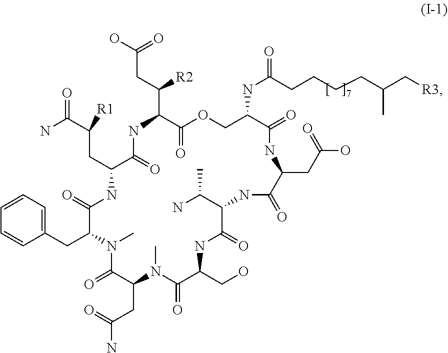
or a pharmaceutically acceptable salt thereof.

7. The PMP inhibitor of claim 1, wherein the PMP inhibitor has the following structure: 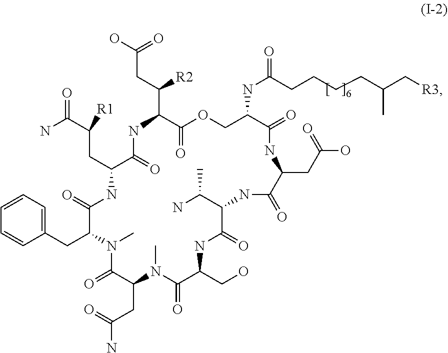
or a pharmaceutically acceptable salt thereof.

8. The PMP inhibitor of claim 1, wherein the PMP inhibitor is selected from a group consisting of:

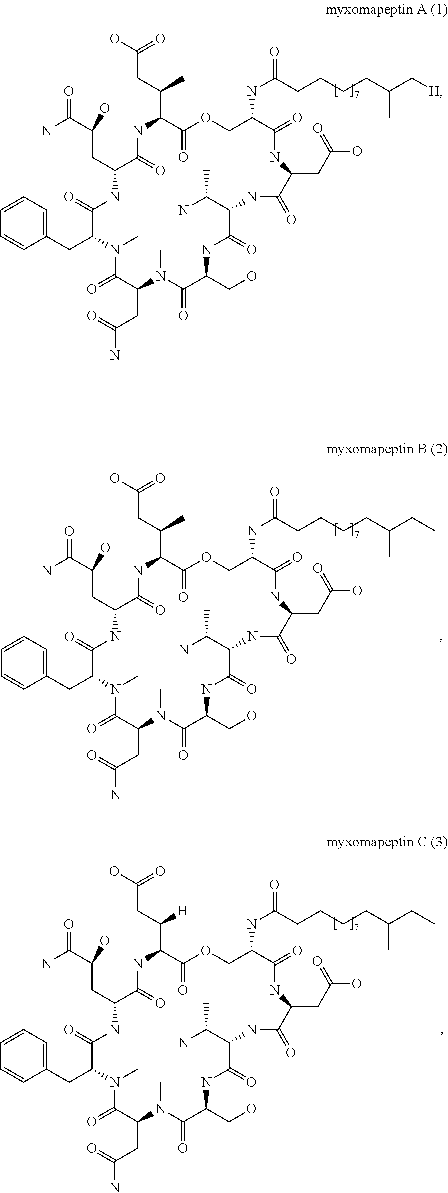

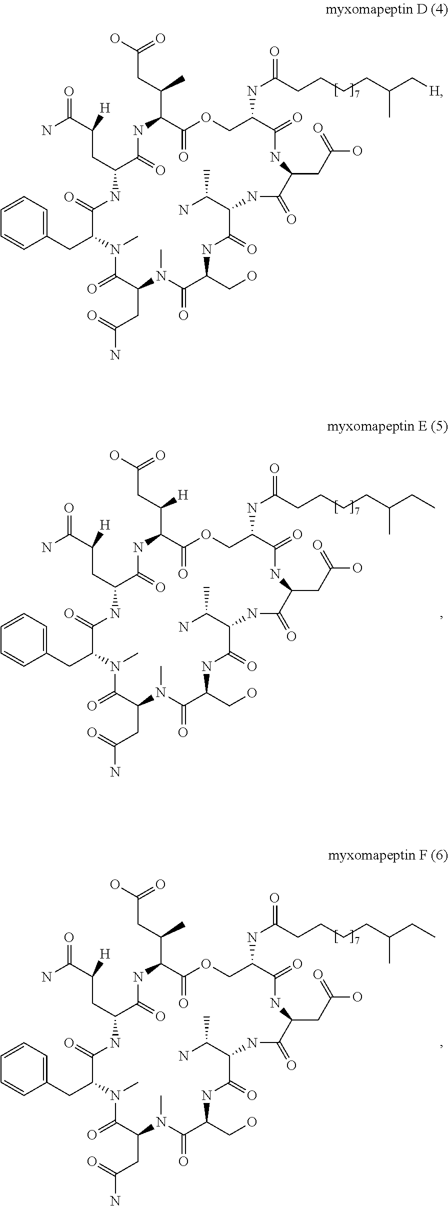

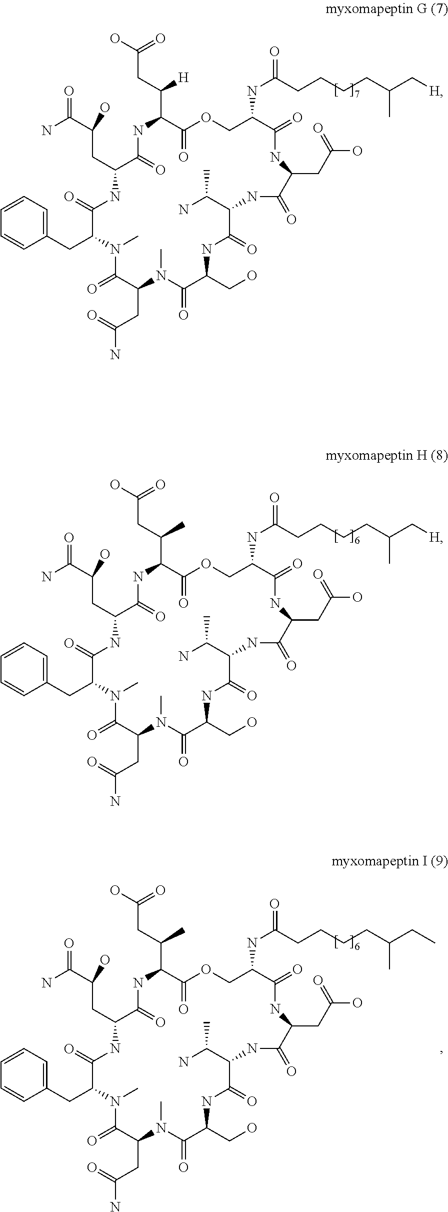

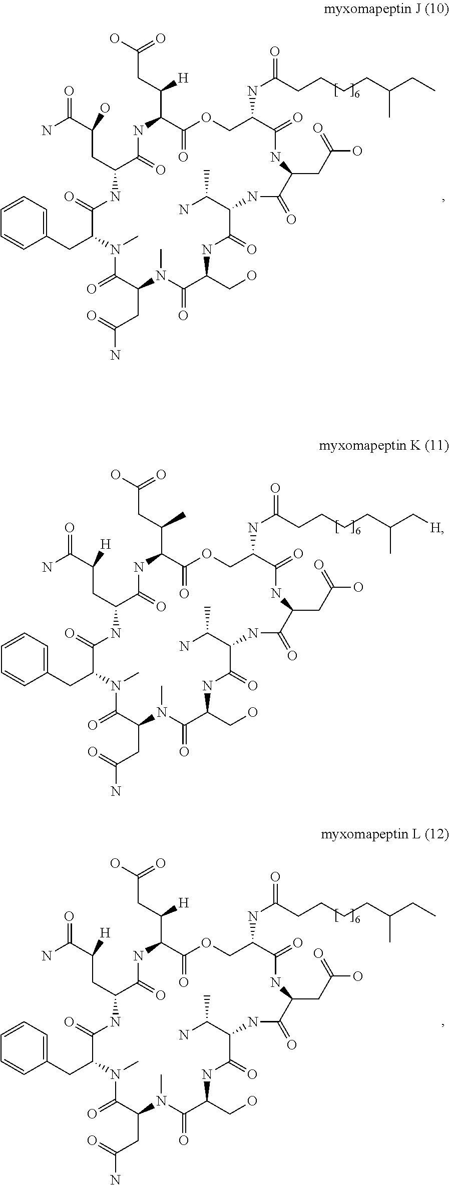

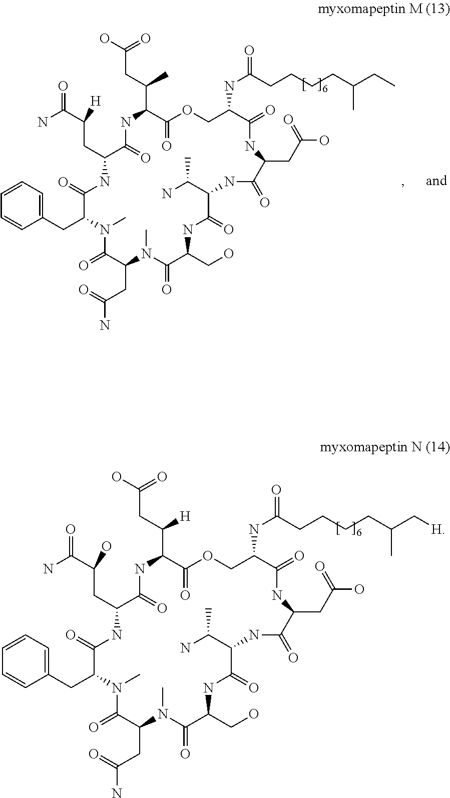

9. A method for treating pseudomyxoma peritonei, the method comprising administering to a subject in need thereof a therapeutically effective amount of a PMP inhibitor of Formula I: 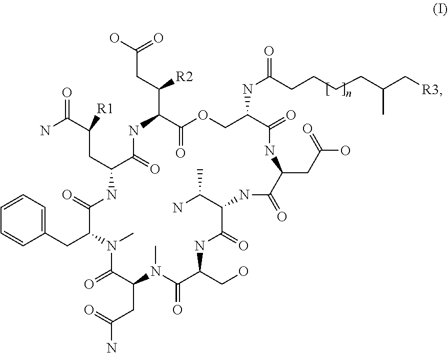
or a pharmaceutically acceptable salt thereof, wherein:
R1 through R3 is independently selected from hydrogen,—OH, or —CH₃; and
n is 6 or 7.

10. The method of claim 9, wherein the PMP inhibitor has the following structure: 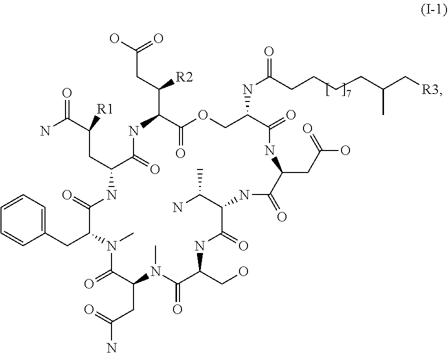
or a pharmaceutically acceptable salt thereof.

11. The method of claim 9, wherein the PMP inhibitor has the following structure: 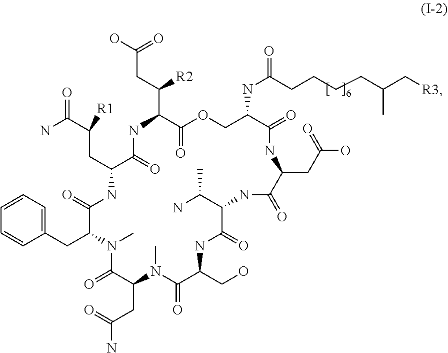
or a pharmaceutically acceptable salt thereof.

12. The method of claim 9, wherein the PMP inhibitor is selected from a group consisting of:

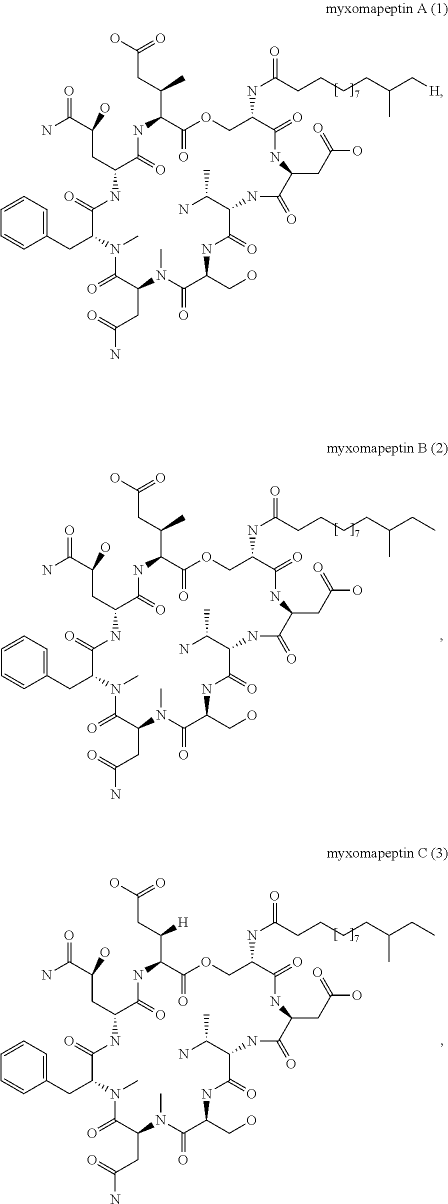

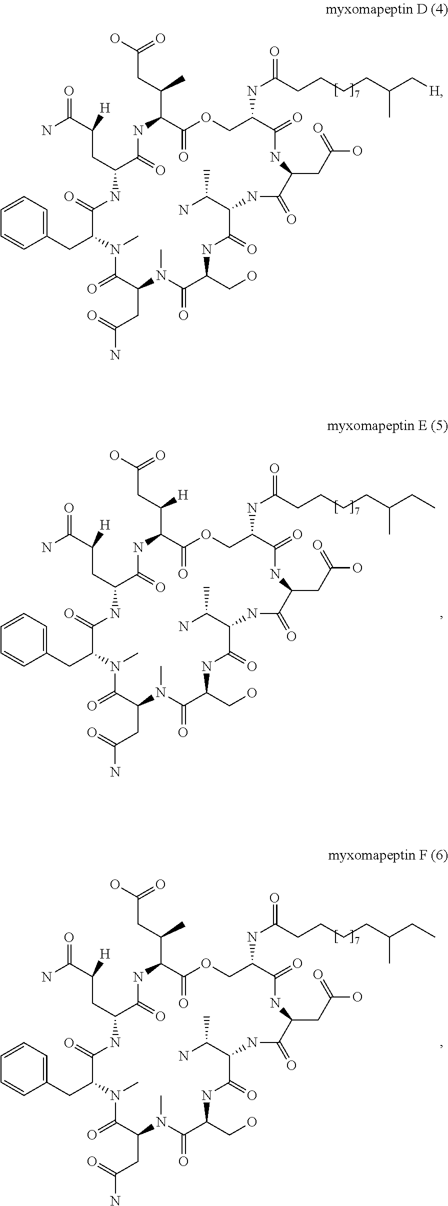

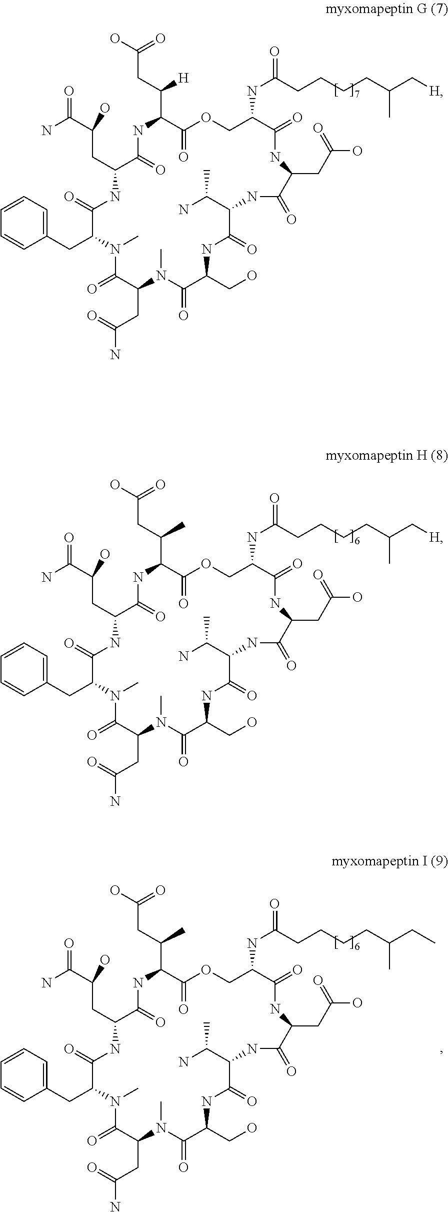

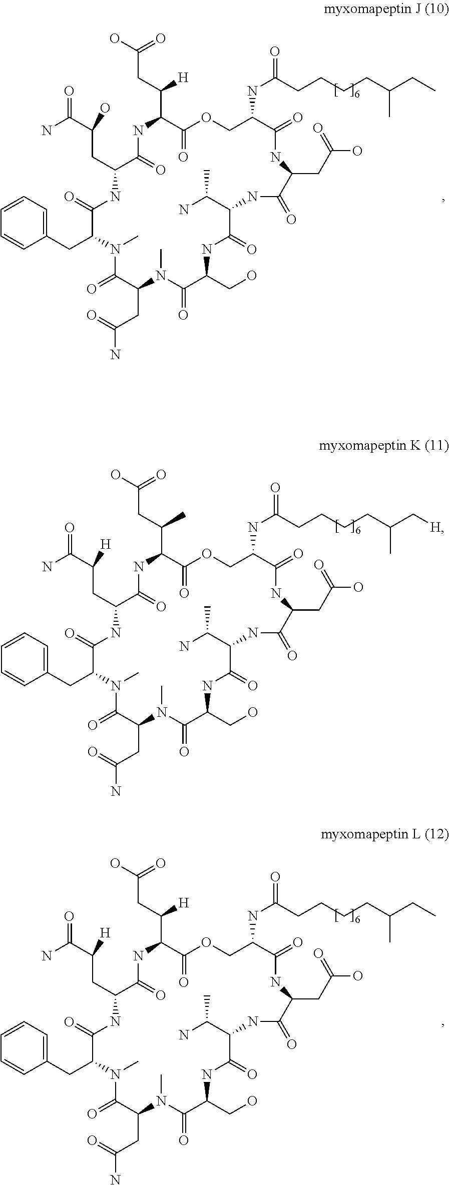

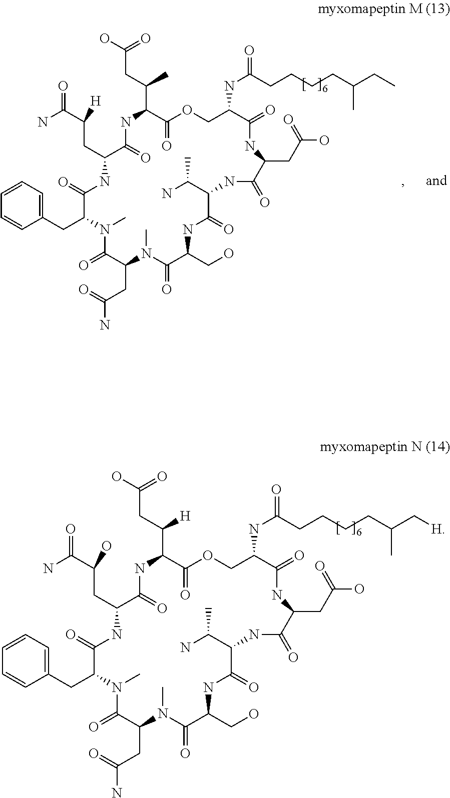

13. The method of claim 9, wherein the PMP inhibitor is administered to the subject at a dose of from about 0.1 μM to about 50 μM.

14. The method of claim 9, wherein the PMP inhibitor is administered to the subject at a dose of from about 1 μM to about 25 μM.

15. The method of claim 9, wherein the PMP inhibitor is administered to the subject at a dose of from about 5 μM to about 20 μM.

16. The method of claim 9, wherein the subject is a cell, a tissue, or a mammal.

17. The method of claim 9, wherein the subject is a human, a non-human primate, a mouse, a rat, or a domesticated mammal.

18. A method for screening and/or identifying an inhibitor for treating pseudomyxoma peritonei, the method comprising:
contacting a stable cell line with an inhibitor;
measuring a level of one or more metabolites selected from Table 1; and
comparing the measured level of one or more metabolites selected from Table 1 with a level of the one or more metabolites in the cell line before contact with the inhibitor,
wherein the presence of, or a significant increase in, the expression level of metabolite after contacting the stable cell line with the inhibitor indicates that the inhibitor effectively treats pseudomyxoma peritonei.

19. The method of claim 18, wherein the cell line is a peritoneal cell line.

20. The method of claim 18, wherein the cell line is PMP498-1, PMP501-1, or ABX023.
